SEQUENCE LISTING (2 sequences)

SEQ ID NO: 1 - DNA, 484 bp, Homo sapiens
ggcgttgccgctctgaatgccagccctaacccctaatgtccctactgcagcctcccagcatcccccctgcaacctcccagcaactccctgtacccctcctaggatcgctcctgcatcccccattatcccccccttcactcctcgcggcatcccccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggcgtctaggtggcaccagaatcccgcgcggactccacccgctgggagctgccctcgcttgcccgtggttgtccagctcagtccctc

SEQ ID NO: 2 - DNA, 528 bp, Homo sapiens
ggcgttgccgctctgaatgccagccctaacccctaatgtccctactgcagcctcccagcatcccccctgcaacctcccagcaactccctgtacccctcctaggatcgctcctgcatcccccattatcccccccttcactcctcgcggcatcccccctgcaccccccagcatcccccctgcagcccccccagcatctcccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggcgtctaggtggcaccagaatcccgcgcggactccacccgctgggagctgccctcgcttgcccgtggttgtccagctcagtccctc

(19) United States
(12) Patent Application Publication
(10) Pub. No.: US 2004/0029138 A1
(43) Pub. Date: Feb. 12, 2004

(54) Global electronic medicine response profile testing network

(76) Inventors: Claire Judith Allan, Stevenage (GB); Dirk E Pollett, Mechelen (BE)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/344,292

(22) PCT Filed: Aug. 9, 2001
(86) PCT No.: PCT/GB01/03624

(51) Int. Cl. C12Q 1/68; G06F 19/00; G01N 33/48; G01N 33/50

(52) U.S. Cl. 435/6; 702/20

(57) ABSTRACT

The present invention relates to healthcare management and specifically to an Internet-facilitated method of improving and applying knowledge of how the genetic make-up of a person affects their response to drug therapy. More particularly, the invention provides a method of using biological markers for the development and prescribing of medicines, such method comprising the steps of obtaining a biological sample from a patient; delivering the sample to a centralized analysis and storage facility; genotyping the sample at the facility, electronically providing the genotype analysis back to said patient upon request by said patient or said patient's healthcare provider in order to enable said healthcare provider to form a judgement as to the most appropriate drug to administer to said patient in view of said patient's genotype; contemporaneously electronically providing the genotype analysis to a peer review body for data analysis and then transmitting such analyzed data to a database so as to enable discovery of one or more associations between a given genotype and a given response to a given drug.

### FIELD OF THE INVENTION

The present invention relates to the broad field of healthcare management and specifically to an Internet-facilitated method of improving and applying knowledge of how the genetic make-up of a person affects their response to drug therapy.

### BACKGROUND OF THE INVENTION

Drug discovery and development is changing at a rapid rate. Pharmaceutical companies are adopting key enabling technologies learned from genetics and genomics to streamline the target identification and validation process. Validated targets are valued from a commercial perspective as early as possible to ensure that medicines are delivered to the market with superior product profiles. Pharmaceutical companies recognise the need to continuously work to improve both the efficacy and safety of drug prescribing. One activity that can help with drug safety and efficacy is pharmacogenetics, which has gained enormous momentum with recent advances in molecular genetics and output from the Human Genome Project.

Pharmacogenetics has the potential to change the way that medicines are prescribed and patient healthcare is managed. Genetics and Genomics will identify individual profiles of markers (alleles) that affect many of the factors that regulate drug response. These include drug absorption, drug distribution, drug metabolism, effective drug concentration and the drug targets themselves, such as receptors and transporters. It will be possible to develop these marker (allelic) profiles into medicine response profile (MRP) tests, which can be used to enable healthcare providers to make optimal medical decisions. Medicine response profiles will not only differentiate patients who will respond to a medicine, they will also identify adverse events and will have the potential to be incorporated into efficacy profiles.

In the short-term (2-5 years) we envisage that MRP testing strategy will focus on genetic markers, such as single nucleotide polymorphisms (SNPs). This is for the simple reason that current technologies are providing such markers at an extremely rapid rate. However, the MRP strategy of the present invention is a fundamental change in how patient healthcare management will be delivered, which is therefore not to be limited solely to use of SNPs as the markers of choice for the practice of the invention. The foremost object of the present invention is the development of MRP's which test for panels of gene-based markers, such as, but not limited to, SNPs, insertion-deletion polymorphisms or mutations, or gene duplications. Another object of the MRP strategy of the present invention is that the concept of MRP testing will be applicable to any type of pharmacogenetic patient testing in the longer term. This would apply to MRP tests, which study marker panels derived from DNA, RNA and/or protein either alone or in different combinations. Pharmacogenetic testing dictates that the MRP tests must be used in combination with administration of a therapeutic agent since the purpose of the test is to obtain safety or efficacy information prior to administration of the therapeutic. MRP testing is a burgeoning concept within the pharmaceutical and biotech industry. Whilst different groups in the pharmaceutical and biotechnology industries are thinking about how best to implement pharmacogenetic testing, no one that we are aware of has proposed a global MRP testing strategy like the one described and claimed in the present invention.

An additional feature of the present invention is a centralised testing facility, which is cost-effective for both patient and healthcare provider and affords the medical community the critical information resource necessary to make optimal decisions for individualized therapeutic regimes.

### SUMMARY OF THE INVENTION

In summary, the invention is a method of using biological markers for the development and prescribing of medicines, such method comprising the steps of obtaining a biological sample from a patient; delivering the sample to a centralized analysis and storage facility; genotyping the sample at the facility, electronically providing the genotype analysis back to said patient upon request by said patient or said patient's healthcare provider in order to enable said healthcare provider to form a judgement as to the most appropriate drug to administer to said patient in view of said patient's genotype; contemporaneously electronically providing the genotype analysis to a peer review body for data analysis and then transmitting such analyzed data to a database so as to enable discovery of one or more associations between a given genotype and a given response to a given drug; and optionally contemporaneously electronically providing the reviewed data and/or the discovered associations back to the facility; and contemporaneously electronically providing the reviewed data and/or discovered associations to one or more healthcare providers upon request by a healthcare provider in order to enable said healthcare provider to form a judgement as to the most appropriate drug to administer to a given patient having a genotype that is present in one or more of said discovered associations.

### DETAILED DESCRIPTION OF THE INVENTION

Definitions

The term “biological sample” shall mean a sample of any tissue from a person. A preferred biological sample is a blood sample

The term “centralised analysis and storage facility” shall mean one or more facilities that are remote from the place that a sample has been gathered. Such a facility uses a standardised, high throughput (rapid) method of biological sample analysis to determine genotype. Such a facility also has adequate computational means to review and validate such data. If there are more than one facility, then they are connected electronically so as to enable electronic transmission of data via the Internet or via another telecommunication network. Additionally, such a facility may have adequate computational means to enable the observation of associations between genotypes and phenotypes.

The term “contemporaneously” shall mean that an electronic transmission of data is taking place or capable of taking place as soon as the data has been compiled or created as the result of operation of an algorithm, and the data has been made available for electronic transmission, and a request has been made for the retrieval and/or transmission of such data.

The term “electronically providing” shall mean the transmission of data via telecommunication means, including telephone data, voice and or fax lines, satellite transmissions, coaxial cable lines, suitable electromagnetic wavelength transmissions and the like.

The term “genetic” shall mean the study of the inheritance of phenotypic traits.

The term “genotype” shall mean the information that resides in the genetic information (DNA sequences) and any derivative information thereof, such as, but not limited to, RNA, and protein and/or gene-expression regulation factors influenced by environmental factors, such as, but not limited to, food intake, concurrent medication, and stress factors.

The term “genotyping” shall mean the determination of the nucleic acid sequences and/or genes to be found in a biological sample of interest. As used herein, “genotyping a subject (or DNA sample) for a polymorphic allele at a defined genomic locus” or “determining the genotype at a polymorphic allelic site” also means detecting which forms of the allele are present in a subject (or a sample). As is well known in the art, an individual may be heterozygous or homozygous for a particular allele. More than two forms of an allele may exist, as is the case with microsatellite markers; thus there may be more than three possible genotypes.

The term “genomic” shall mean the study of the effects of alteration of nucleic acid sequence upon phenotype.

Ther term “healthcare provider” shall include, but not be limited to, physicians, nurses, physician assistants, medics, child health associates, nurse practitioners, dentists and pharmacists.

The term “medical data card” shall mean an electronic data storage device with built-in security access designed to provide fast access and/or store an individual's medical records.

The term “peer review body” shall mean any number of individuals qualified by education, experience and training, to review genetic data for completeness, quality, validity and/or for the observation of associations between incidence of a genotype of interest and a phenotype of interest.

The term “phenotype” shall mean any given physical, biochemical, or physiological state, status or condition of an individual or population of individuals as determined genetically, up to and including the entire makeup of a given individual or population of individuals. Phenotypes can include the outcome(s) of administration of a given drug to a given individual, including efficacy results and including adverse event results.

Sample Acquisition

A single biological sample is collected from a patient while that patient is in a healthcare provider's office or clinic. Geographic location of the office or clinic has no effect on the operation of the method, making the method globally applicable. Preferably this is the patient's first visit to the healthcare provider, and more preferably, the patient is asymptotic at that time. Conversely, that specific healthcare provider's office or clinic would collect such samples for MRP testing from all patients that use that clinic, who would consent to providing a sample for MRP. Once a biological sample has been collected and has been stored in a central repository, the MRP testing becomes truly applicable on a world-wide scale and sufficiently fast, provided that an Internet link is available.

If the first sample is collected from a patient while they are asymptotic, this sample may be used later as an individual's reference material for determining the individuals healthy baseline reference value for biochemical markers of disease. Therefore, rather than using the reference value of a population to determine if a biochemical marker of disease is increased or decreased, the individual's own healthy baseline reference value could be used.

The patient's biological sample would be transported to and stored in a safe repository at a centralised laboratory testing facility. Biological components contained within the sample (DNA, RNA, protein, specific cell types and so forth) will be isolated, quality controlled and stored at the repository under strictly regulated conditions.

Requesting an MRP Test

Upon a patient's subsequent visit to a healthcare provider, either for a follow-up consultation or with a specific concern or illness, the healthcare provider would request an MRP on-line for the sample in the central laboratory via an on-line request while the patient is waiting there at the healthcare provider's office or clinic. MRP information will only be generated on biological samples for which the corresponding individual has given informed consent. The MRP information will only be released from the secured database upon the specific consent of the patient. Once the test has been requested, it will be conducted by the central facility and returned to the healthcare provider/healthcare provider preferentially, ideally within 30 minutes. Alternatively, the central testing facility could test all incoming samples for all available MRP profiles and store the results in a secure database ready for access upon the healthcare provider/healthcare provider's request. Additional MRP tests will be run routinely as new MRP profiles become available and the data will be added to existing databases for future reference. The test result can furthermore be stored onto a machine readable medical data card that the patient can then take with them to another healthcare provider for data retrieval in the future. The patient's medical data card will be used by all healthcare providers/healthcare providers who treat that patient, thereby collecting information that has been requested by multiple healthcare providers.

Test Result Interpretation

Whenever so authorised, the MRP result data is accessed by the healthcare provider, who is then able to correlate the patient's MRP with what is known about prescribing the most appropriate drug for a given genotype. The variation in patient response rate to a drug, or the occurrence of adverse events following administration of a drug to certain patients, is mainly due to variation in individual patient's genetic background (genotypes). A drug which is known to have a high response rate with little or no risk of an adverse event for a population of patients having a certain genotype, may have a significantly decreased response rate or even show a risk of causing an adverse event in another population of patients who have the same disease but who have a different genotype for the target. Prepared with the knowledge of the patient's genotype by virtue of the MRP, and being able to access the body of knowledge correlating varying genotypes with different outcomes, the healthcare provider can make an informed decision and provide the right drug to the right patient and obtain a favorable therapeutic outcome.

Regulation of MRP Testing

A key feature of the method of the invention is that the patient would have control over access to the sample. Regardless of where the patient is in the world, they can request an MRP test from any healthcare provider using the patient's medical data card which links into the Internet infrastructure, and which manages MRP testing. One potential embodiment of the invention would accomplish this by attaching a unique label, such as for example, a bar code or hologram to the initial biological sample (for instance a blood sample), that would correlate to a bar code on the patient's medical data card. The patient's medical data card would be needed for the healthcare provider to access the stored data, which the patient would hand over to the healthcare provider for that limited purpose. If required, an added level of security can be put in place such that the healthcare provider has to enter a second card into a reader to enable an information update to be made to the patient's record or to request the MRP test. Thus, a patient could control access to the data, and likewise enable any healthcare provider at any place in the world at any time to access the data on behalf of the patient. As an additional feature of the invention, the card could be used to track the amount of biological sample available for testing, initiating a sample request when the amount of stored biological sample is below a logistical threshold. Any biological sample taken for MRP testing should not be used for any other genetic testing purposes other than for the ones for which consent was given when the sample was obtained.

Centralised Testing Facility

It is a feature of the present invention that biological samples can be collected and acquired from any geographical location throughout the world. All samples will be collated at a few Centralised sites connected via the Internet to their regional locations. Biological sample and data storage would take place at one or more centralized storage facilities. The facilities would be connected electronically via the Internet using an inspected and authorized service provider. Via the Internet, the facilities would receive data from outlying healthcare provider offices or clinics, transmit data to the offices and clinics (when so authorized by the patient by means of their medical data card), transmit data to other storage facilities, and transmit data to one or more pharmaceutical companies for drug discovery and development purposes (if such data had been anonymised after informed consent of the patient made it permissible to do so)

Pharmaceutical Industry Consortia

In a preferred embodiment of the method of the invention, a consortium of pharmaceutical companies is organized to create a global, centralized repository of biological data that is maintained and updated by data downloads from all consortium members, and that is conversely available for (anonymised) data retrieval by all consortia members for the purposes of drug discovery and development, and for the purpose of healthcare management. Other potential consortia members can include regulators, patient advocacy groups, and insurers and diagnostics manufacturers. The global network of central laboratory facilities will ensure that legal and ethical standards are maintained, will promote sample security and will reduce the risk for misuse of patient information. Using a consortium will streamline reimbursement for payers and alleviate the need for profit margins on the manufacture and sale of diagnostic kits.

The use of one or more centralized laboratory and storage facilities eliminates the need for marketing, sales and distribution of specific diagnostic kits, thereby minimizing the cost per test. Minimizing costs is made possible, thereby making MRP-testing acceptable to payers, since such a centralized facility will be using high throughput rapid result analytical devices for the generation of data from tissue samples, thereby creating a clear cost/benefit ratio.

Community Benefits

The healthcare community as a whole, including both healthcare providers and pharmaceutical companies, would greatly benefit from being able to access anonymised patient biological data in their drug discovery and development activities. Such data enable researchers to locate disease susceptibility genes, locate targets for drug intervention in disease processes, and accumulate enough data to discover associations between certain genotypes and diseases, or between certain genotypes and responses to drugs. Each of these key drug discovery activities would be much more rapidly enabled by the invention, since an enormous global pool of diverse patient populations would be transferring MRP test data to the data storage centers, which would be analyzing the data on a real-time basis to generate genotype-phenotype associations, making the data and the analyses available in real time to drug researchers, and subsequently making the association knowledge available in real time back to healthcare providers to complete the loop. Healthcare providers would be able to make use of the experience that all of their colleagues have had in treating patients and diseases similar to the one for which they need to prescribe medication. The proposed MRP-testing procedure will allow the collection of information about patients' phenotype and drug response. This information could, after peer review, be made available to all healthcare providers to improve and facilitate their decision making for therapeutic intervention and effective disease management. In addition, the proposed MRP-testing procedure would allow adverse event recording post-marketing of a new drug on a significant scale. Should adverse events be picked up, once a therapeutic has come onto the market, a reference database analyzing existing patient's data, including adverse event reporting would help to identify the seriousness of the problem. Prompt identification of adverse events and peer review of the severity of the effect will help to determine the best course of action. The best action may be, but is not limited to, a simple label change or drug reformulation or renewed market positioning. At its most extreme, an adverse event may dictate that the best course of action is removal of a drug from the market.

Further cost reductions are possible with an effective adverse event recording system. One possibility is that pharmaceutical companies will be able to convince regulators to reduce the size of phase three human clinical trials since phase four trials and studies can be well controlled and followed up using the method of the invention. If costs of clinical trials can be reduced, there will be more incentive for drug companies to develop drugs with a smaller market potential but for which there is a significant unmet medical need, or where there is a need for an increase in patient compliance.

Regulatory Submissions

An additional object of the present invention would be to enable new standards of regulatory agency approval for new human drugs that would require submission of data which may tend to identify patients as responders, non-responders or likely sufferers of adverse events, where such data was obtained through practice of the invention as claimed. Again, reducing costs of clinical trials provides the incentive for drug companies to develop drugs with a smaller market potential but for which there is a significant unmet medical need or need for improved likelihood of compliance.

Sample Collection and Storage Systems

In order to achieve maximum utilization of the biological information that is to be found in a patient's biological sample, a standardized sample collection and storage procedure must be used. Many processes and systems exist for such systematic collection and storage, and these are well known to those of ordinary skill in the fields of clinical management, information systems, and management of clinical trials of experimental human drugs.

Technologies for MRP Testing

Ideally, the method of the invention should use a testing technology that will: take thirty minutes or less or be available in real time on a database; require minimal sampling from the patient; enable availability of the test results to the patient and healthcare provider while the patient is still in the same consultation visit with the healthcare provider; and comply with all applicable international standards and regulations on the storage and testing of samples.

Data Management Systems

Under the method of the invention, MRP testing data is to be continually collected, accessed and assessed via a centralized testing facility. Communications to and from distant locations and computers will be to the centralized testing facility by means of the International Network of Computers (the Internet). Internet communications software, data management software, database software, bioinformatic software, clinical modeling software, are all needed for the method of the invention, and are all readily commercially available. Such availability and applicability, and the operation of such software are all well known to those of ordinary skill in the fields of clinical management, telecommunications, information systems, and management of clinical trials of experimental human drugs.

Payment and Reimbursement Systems

In the most preferred embodiment of the method of the invention, whenever a healthcare provider orders a test for a given patient, the test is ordered in real time from the healthcare provider's computer, and the cost of the MRP test is included in the price of the pharmaceutical and is not billed separately to the patient.

### EXAMPLES

The practice of the method of the present invention is additionally facilitated by the following descriptions of the conduct of a pharmacogenetic studies, which includes descriptions of how to conduct and interpret the various phases of the study, and the technologies underlying the conduct of the study.

### Example 1

### Polymorphisms of the 5-hydroxytryptamine Transporter Gene

Studies were conducted that relate to polymorphisms in the 5-hydroxytryptamine transporter (5-HTT) gene, and phenotypes that are associated or correlated therewith. More particularly, the studies related to the correlation of such polymorphisms to the response of subjects with gastrointestinal disorders (such as Irritable Bowel Syndrome (IBS)) to pharmaceutical treatment. Such studies further related to methods of screening compounds for pharmaceutical activity. The present studies also relate to methods of genotyping subjects for predictive purposes, again, based upon said correlations.

Many gastrointestinal disorders of unknown etiology, including Irritable Bowel Syndrome (IBS), are believed to be multifactorial disorders. In many of these disorders, no biochemical marker has been found and diagnosis is accomplished primarily by observation of clinical symptoms. Unlike single gene Mendelian disorders, complex disorders such as diabetes, migraine and cardiovascular disease tend to be multifactorial and are caused by the interaction of one or more susceptibility genes with environmental factors. To date, no individual susceptibility genes for IBS have been identified by either linkage or association studies.

Irritable bowel syndrome (IBS) is a common gastrointestinal disorder characterized by abdominal pain and discomfort, and altered bowel habit. IBS may be characterized by symptoms of either constipation or diarrhea, or alternating constipation and diarrhea. Currently, there are no single pathophysiological or diagnostic markers of IBS. However, various diagnostic criteria for IBS are available, e.g. Thompson et al., Gastroent. Int., 2:92 (1989); Manning et al., Br. Med. J. 2:653 (1978); Thompson et al., Gut 45:1143 (1999)

Antagonism at 5-hydroxytryptamine receptors, such as by alosetron hydrochloride, has been shown to be useful in the treatment of diarrhoea-predominant irritable bowel syndrome.

Alosetron hydrochloride (CAS registry number: CAS-122852-69-1; see U.S. Pat. No. 5,360,800, the entire disclosure of which is incorporated herein by reference) is a 5-HT3 receptor antagonist. Both animal and human studies indicate that 5-HT3 receptor blockade has therapeutic value in the treatment of irritable bowel syndrome, particularly in diarrhea-predominant IBS. (The disclosures of all US patents cited herein are incorporated herein by reference in their entirety.)

In double blind, placebo controlled studies, alosetron hydrochloride has been shown to reduce pain and improve bowel function in patients with Irritable Bowel Syndrome (IBS). See Bardhan et al., Aliment Pharmacol Ther 2000 January; 14(1): 23-34; Jones et al., Aliment Pharmacol Ther 1999 Nov; 13(11): 1419-27; Camilleri et al., Aliment Pharmacol Ther 1999 Sep; 13(9): 1149-59; Mangel et al., Aliment Pharmacol Ther 1999 May; 13 Suppl 2:77-82. Alosetron has further been indicated as a potential treatment for the symptomatic relief of carcinoid diarrhea. Saslow et al., Gut 1998 May; 42(5): 628-34.

5-hydroxytryptamine (5HT) receptors have been identified and characterized in the gastrointestinal tract, including 5HT3, 5HT4, and 5HT1a receptors; these receptors are involved not only in modulating gut motility but also in visceral sensory pathways. Various 5HT3 antagonists (e.g., alosetron, granisetron and ondansetron) have been identified for the treatment of IBS. This class of drug appears to reduce visceral sensitivity and have inhibitory effects on motor activity in the distal intestine. Full and partial 5HT4 agonists (e.g., HTF919, tegaserod) are potential therapeutics to improve constipation-predominant IBS. Preliminary studies suggest that these agents may have therapeutic potential in IBS. Farthing et al., Baillieres Best Pract Res Clin Gastroenterol. 1999 October; 13(3): 461-71. 5HT4 antagonists (piboserod, SB-207266A) have also been suggested for the treatment of IBS.

The human 5HTT protein is encoded by a single gene (SLC6A4) found on chromosome 17q12 (Ramamoorthy et al., Proc. Natl. Acad. Sci. USA 90:2542 (1993); Gelemter et al., Hum. Genet. 95:677 (1995); Lesch et al., J. Neural Transm. 91:67 (1993). The 5HT Transporter regulates the magnitude and duration of serotonergic responses. An insertion/deletion polymorphism consisting of a 44 base pair segment in the transcriptional control region 5′ upstream to the 5HTT coding sequence has previously been identified. The deletion (or short) allele of this polymorphism is associated with decreased transcription efficiency of the 5HTT gene promoter, decreased gene expression, and decreased 5-hydroxytryptamine uptake. (Heils et al., J. Neural Transm. 102:247 (1995); Heils et al., J. Neurochem 66:2621 (1996), Lesch et al., Science 274:1527 (1996)). Additionally, various biochemical studies suggest that 5HT uptake function is frequently reduced in psychiatric illnesses, and variation in functional 5HTT expression due to 5HTT promoter polymorphism has been implicated as a potential genetic susceptibility factor for affective disorders (Collier et al., Mol Psychiatry 1996 December; 1(6): 453-60; Lesch et al., Science Nov. 29, 1996; 274(5292): 1527-31; Furlong et al., Am J Med Genet Feb. 7, 1998; 81(1): 58-63; Menza et al., J Geriatr Psychiatry Neurol 1999 Summer; 12(2):49-52; and Rosenthal et al., Mol Psychiatry 1998 March;3(2):175-7.

It has been determined that polymorphisms in the 5-hydroxytryptamine transporter (5HTT) gene are correlated with the response of subjects with IBS to pharmaceutical therapy More particularly, it was found that an insertion/deletion polymorphism in the 5′ non-coding region of the 5HTT gene is a predictor for the response of patients with IBS to treatment with a 5HT antagonist; and there was identified a genetic subset of IBS patients that displays a higher incidence of relief of IBS symptoms and a lower incidence of the side effect of constipation when treated with alosetron (compared to patients with an alternative polymorphism at the same site of the 5HTT gene).

Consequently, these observations led to a method of screening a patient population to identify those subjects with an increased likelihood of responding favorably to treatment with a 5HT antagonist for a gastrointestinal disorder. The subjects may have been previously diagnosed as having IBS, or the screening may be used in conjunction with IBS diagnostic efforts.

A further aspect is a method of screening a subject suffering from a gastrointestinal disease that is treatable with a 5-hydroxytryptamine (5HT) ligand, as an aid in predicting the subject's response to treatment with a 5HT ligand. The method comprises obtaining a sample of the subject's DNA and determining the genotype of the subject at a polymorphic allelic site in the 5hydroxytryptamine transporter (5HTT) gene, where different genotypes at that site have been associated with different incidences of a phenotypic response to treatment with a 5HT ligand. The genotype that is detected in the sample indicates that the subject is likely to have the phenotypic response associated with that genotype.

Another aspect is a method of screening a subject with irritable bowel syndrome (IBS), as an aid in predicting the subject's response to treatment with a 5HT ligand. The method comprises obtaining a sample of the subject's DNA and determining the genotype of the subject at a polymorphic allelic site in the 5hydroxytryptamine transporter (5HTT) gene, where different genotypes at that site have been associated with different incidences of a phenotypic response to treatment with a 5HT ligand.

A further aspect is a method of screening a 5-hydroxytryptamine (5HT) ligand for variations in a measurable phenotypic effects among genetic subpopulations of subjects with a gastrointestinal disorder. The method comprises administering the 5HT ligand to a population of subjects suffering from the gastrointestinal disorder, and obtaining DNA samples from each of the subjects. The DNA samples are genotyped for a polymorphic allele of the 5-hydroxytryptamine transporter (5HTT) gene, and correlations between the polymorphic allele genotype and the occurrence of a phenotypic response in the population of subjects are determined. Detection of a genotype that is correlated with an increased or decreased incidence of a desired therapeutic response or a side effect (compared to the incidence in subjects with alternative genotypes) indicates that the effectiveness of the ligand in treating that gastrointestinal disorder varies among genetic subpopulations.

Genetic samples were obtained from subjects enrolled in clinical trials of alosetron for the treatment of IBS. The genetic samples were screened for an insertion/deletion polymorphism in the 5′ non-coding region of the 5-hydroxytryptamine transporter gene (5HTT gene), using polymerase chain reaction (PCR) technology. The alleles were labeled as “del” (deletion) or “ins” (insertion) resulting in three possible genotypes (del/del; del/ins or ins/ins). The insertion polymorphism (allele “ins”) had SEQ ID NO:2:

| (SEQ ID NO:2) | |
|---|---|
| ggcgttgccg ctctgaatgc cagccctaac ccctaatgtc cctactgcag cctcccagca | 60 |
| tcccccctgc aacctcccag caactccctg tacccctcct aggatcgctc ctgcatcccc | 120 |
| cattatcccc cccttcactc ctcgcggcat cccccctgca ccccccagca tcccccctgc | 180 |
| agccccccca gcatctcccc tgcaccccca gcatcccccc tgcagccctt ccagcatccc | 240 |
| cctgcacctc tcccaggatc tcccctgcaa cccccattat cccccctgca cccctcgcag | 300 |
| tatcccccct gcacccccca gcatcccccc atgcaccccc ggcatccccc ctgcacccct | 360 |
| ccagcattct ccttgcaccc taccagtatt cccccgcatc ccggcctcca agcctcccgc | 420 |
| ccaccttgcg gtccccgccc tggcgtctag gtggcaccag aatcccgcgc ggactccacc | 480 |
| cgctgggagc tgccctcgct tgcccgtggt tgtccagctc agtc cctc | |

Legend:
PCR primer sequences are in underlined typeface
Non-coding sequences are shown in lowercase typeface
Polymorphic bases are shown in bold typeface
Base numbering is relative to the sequence shown
Polymorphism numbering is relative to the gene cDNA sequences

The “del” allele represents a deletion of approximately 44 base pairs in the 5′ untranslated region of the 5HTT gene. This deletion in the transcriptional regulatory region has been associated with decreased re-uptake of 5HT and therefore an increased 5HT basal level. Therefore, the del/del genotype is postulated to result in a lower transcription efficiency, lower production of 5HTT, and reduced basal 5HT re-uptake (compared to the del/ins or ins/ins genotype). The del/del, del/ins and ins/ins genotypes were approximately evenly distributed among the subjects. Of 219 subjects, 71 were del/del 5HTT; 75 were del/ins 5HTT; and 73 were ins/ins 5HTT.

It was further determined that the del/del genotype is associated with an increased incidence of relief of IBS symptoms and a lower frequency of constipation as an effect of treatment with a 5HT3 antagonist, and therefore an increased incidence of favorable therapeutic response to treatment with a 5HT3 antagonist (compared to subjects with the del/ins or ins/ins genotype treated with the same 5HT3 antagonist).

In each of the three 5HTT genotypes alosetron was more effective than placebo in relieving IBS symptoms. However, in the del/del genotype group (homozygous for the deletion polymorphism), the incidence of relief of IBS symptoms for both alosetron and placebo was increased compared to other 5HTT genotypes. Subjects with the del/del genotype also showed a reduced incidence of constipation compared to the del/ins and ins/ins 5HTT genotype groups. Subjects with the del/del 5HTT genotype showed an increased incidence of favourable therapeutic response with a higher incidence of relief of IBS symptoms and a lower incidence of the alosetron-induced side effect of constipation, when compared with subjects who had del/ins or ins/ins 5HTT genotypes.

Accordingly, a subject who suffers from a gastrointestinal disease that is treatable with 5HT ligands can be genetically screened, to aid in predicting their response to such treatment. Screening comprises obtaining a sample of DNA from the subject and screening the DNA to determine the genotype (presence/absence of polymorphic alleles) at a predetermined polymorphic site in the 5hydroxytryptamine transporter (5HTT) gene, where different genotypes at that site have previously been associated with different incidences of a phenotypic response to treatment with a 5HT ligand. The presence of a particular genotype therefore indicates an increased likelihood that the individual subject will exhibit the associated phenotype. The genotype will rarely be absolutely predictive, i.e., where a population with a certain genotype displays a high incidence of a particular phenotype, not every individual with that genotype will display the phenotype. However, it will be apparent to those skilled in the art that genotyping a subject as described herein will be an aid in predicting the response a subject will have to treatment with a 5HT ligand, and thus assist in the treatment decision.

As used herein, “genotyping a subject (or DNA sample) for a polymorphic allele at a defined genomic locus” or “determining the genotype at a polymorphic allelic site,” means detecting which forms of the allele are present in a subject (or a sample). As is well known in the art, an individual may be heterozygous or homozygous for a particular allele. More than two forms of an allele may exist, as is the case with microsatellite markers; thus there may be more than three possible genotypes.

As used herein, a subject that is “predisposed to” a particular phenotypic response based on genotyping of a polymorphic allele will be more likely to display that phenotype than an individual with a different genotype at that polymorphic allele. Where the phenotypic response is based on a biallelic polymorphism, the response may differ among the three possible genotypes (Eg. For 5HTT: del/del, del/ins and ins/ins).

As used herein, a “genetic subset” of a population consists of those members of the population having a particular genotype. In the case of a biallelic polymorphism, a population can potentially be divided into three subsets: homozygous for allele 1, heterozygous, and homozygous for allele 2.

As used herein, a gastrointestinal disease treatable with 5HT ligands' is one in which the administration of a 5HT ligand (in an appropriate pharmaceutical formulation, and in a therapeutically effective amount) has been shown to reduce or alleviate symptoms, without causing unacceptable side effects. Such therapeutic effectiveness is typically evidenced by Regulatory Authority (e.g. FDA, EMEA) approval of the pharmaceutical preparation, or by publication of the results of clinical studies in peer-reviewed medical journals. Therapeutically effective amounts of such compounds can be readily determined by those skilled in the art using, e.g., dose-response studies. As used herein, the term ‘5HT ligand’ encompasses antagonists and agonists of 5HT receptors, including partial agonists and drugs that interact with 5HTT (e.g. selective serotonin re-uptake inhibitors, SSRI's). 5HT ligands may bind to any subtype of the 5HT receptor, including 5HT3 and 5HT4 receptors; the ligands may be specific for a particular receptor subtype.

Known 5HT-related compounds include 5HT3 antagonists (e.g., ondansetron, granisetron, tropisetron, dolasetron, mirtazapine, itasetron, pancopride, zatosetron, azasetron, cliansetron, YM-144 (Yamanouchi) and RS17017 (Roche)).

5HT4 agonists are also known, including tegaserod, prucalopride, norcisapride and the 4-amino-5-chloro-2-methoxy-N-(1-substituted piperidin-4-yl) benzamide known as Y-34959 (Yoshitomi Pharmaceuticals), and buspirone. The use of 5HT4 agonists to treat constipation-predominant IBS has been proposed. 5HT4 antagonists include piboserod (SmithKline Beecham).

Dual 5HT3 and 5HT4 agonists include renzapride (SmithKline Beecham) and E3620 (Eisai). A 5HT1a agonist is also known, LY315535 (Eli Lilly).

Selective serotonin re-uptake inhibitors include fluoxetine, etc.

As used herein, a “side effect” is an undesirable response to the administration of a therapeutic compound, i.e., and an effect that is not directed to alleviating the symptoms or cause of the disease being treated. Side effects range from minor inconveniences to more serious events.

In these methods, a compound with 5HT-ligand activity may be screened for variation in its effects among genetic subpopulations of subjects with a gastrointestinal disorder. Such methods involve administering the compound to a population of subjects suffering from a 5HT-mediated gastrointestinal disorder, obtaining DNA samples from the subjects (which may be done either prior to or after administration of the compound), genotyping a polymorphic allelic site in the 5HTT gene, and correlating the genotype of the subjects with their phenotypic responses (both favorable and unfavorable) to the treatment. A genotype that is correlated with an increased incidence of a desired therapeutic response (or a decreased incidence of an undesirable side effect), compared to the incidence in subjects with alternative genotypes at the polymorphic allelic site, indicates that the effectiveness of the compound in treating such gastrointestinal disorder varies among genetic subpopulations.

Stated another way, the method may be used to determine the correlation of a known 5HTT polymorphic allele with the response of subjects with gastrointestinal disorders (such as IBS) to treatment with a 5HT ligand. The population of subjects with the disease of interest is stratified according to genotype for the particular polymorphic allele, and their response to a therapeutic agent is assessed (either prospectively or retrospectively) and compared among the genotypes. The response to the therapeutic agent may include either, or both, desired therapeutic responses (e.g., the alleviation of signs or symptoms) and undesirable side effects. In this way, genotypes that are associated with an increased (or decreased) incidence of therapeutic efficacy, or an increased (or decreased) incidence of a particular side effect, may be identified. The increase or decrease in response is in comparison to the other genotypes, or to a population as a whole. Once this relative increase or decrease has been observed, responders and non-responders can be identified and assigned to separate sub-populations. A non-responder will be a subject displaying a defined degree of decreased incidence of therapeutic efficacy, possibly displaying no therapeutic efficacy at all. Alternatively, a non-responder can be categorized as a subject displaying a defined degree of increased incidence of a side effect of interest, ranging from relatively benign side effects to those that are potentially life-threatening.

Polymorphisms are variant sequences within the human genome that may or may not have a functional consequence. These variants can be used in all aspects of genetic investigation including the analysis and diagnosis of genetic disease, forensics, evolutionary and population studies. Two types of genetic analyses are typically performed: linkage and association studies.

A linkage study provides genetic map information where there is no prior knowledge or assumption about the function of a gene. In a linkage study one uses DNA polymorphisms to identify chromosomal regions that are identical between affected relatives with the expectation that allele sharing frequencies will be higher for a marker (polymorphism) whose chromosomal location is close to that of the disease allele. Physical cloning of a linkage region narrows down the DNA sequence that could harbor the candidate disease gene. While linkage analysis locates the disease locus to a specific chromosome or chromosome region, the region of DNA in which to search for the gene is typically large, on the order of several million base pairs.

In contrast to linkage, association shows the coexistence of a polymorphism and a disease phenotype in a population. Association studies are based upon linkage disequilibrium, a phenomenon that occurs between a marker and a disease phenotype if the marker polymorphism is situated in close proximity to the functional (disease)-causing causing variant. Since the marker and disease-causing variant are in close proximity, it requires many generations of recombination to separate them in a population. Thus they tend to co-exist together on the same chromosome at a higher than expected frequency. A marker (polymorphism) is said to be associated with a specific phenotype when its frequency is significantly higher among one phenotype group compared to its frequency in another. In general, the closer a marker is to the functionally polymorphic site, the stronger the association.

Association studies offer the opportunity to finely map linkage regions, map loci refractory to linkage analysis and map unknown predisposition loci. Polymorphisms that are in linkage disequilibrium with each other can be spaced over large regions. Linkage disequilibrium has been reported in regions as small as 1 kb or as large as 500 kb. Polymorphisms throughout a gene can be in linkage disequilibrium with each other, such that it is valuable to study the whole genome structure—introns, exons, promoters and transcriptional regulatory regions, and 3′ and 5′ untranslated regions. A marker that is in linkage disequilibrium with a functional polymorphism can be used as the basis of a test that correlates that polymorphism with a phenotype of interest.

A polymorphism in the 5HTT gene plays a role in the response of subjects to pharmaceutical treatment of IBS, and thus the genotyping of the 5HT Transporter (5HTT) gene (either directly or via its expression product) is useful in identifying therapeutic compounds with measurable effects that vary among 5HTT genotypes. The effect to be measured will depend on the particular gastrointestinal condition, therapeutic compound, and patient population, as will be apparent to one skilled in the art. The measurable effect may be the relief of, or change in, a pathologic sign or symptom or the occurrence of a side effect related to compound administration. Measurement may be objective or subjective (e.g., by patient self-reporting). The association of a 5HTT genotype with a therapeutic response will provide a method of determining the probability that an individual subject will respond in a particular way to treatment with 5HT ligands. In genotyping, the characteristic that is typically measured is one that can be influenced by a polymorphism in the gene or its expression product. As used herein, the term polymorphism includes Single Nucleotide Polymorphisms (SNPs), insertion/deletion polymorphisms; microsatellite polymorphisms; and variable number of tandem repeat (VNTR) polymorphisms.

Methodologies in the Detection of Polymorphisms

Polymorphic alleles are typically detected by directly determining the presence of the polymorphic sequence in a polynucleotide or protein from the subject, using any suitable technique that is known to those of ordinary skill in the art. Such a polynucleotide is typically genomic DNA, or a polynucleotide derived from this polynucleotide, such as in a library made using genomic material from the individual (e.g. a cDNA library). The processing of the polynucleotide or protein before the carrying out of the method of the invention is further discussed below. Typically the presence of the polymorphism is determined in a method that comprises contacting a polynucleotide or protein of the individual with a specific binding agent for the polymorphism and determining whether the agent binds to the polynucleotide or protein, where the binding indicates that the polymorphism is present. The binding agent may also bind to flanking nucleotides and amino acids on one or both sides of the polymorphism, for example at least 2, 5, 10, 15 or more flanking nucleotide or amino acids in total or on each side. In one embodiment the agent is able to bind the corresponding wild-type sequence by binding the nucleotides or amino acids which flank the polymorphism position, although the manner of binding will be different than the binding of a polymorphic polynucleotide or protein, and this difference will be detectable (for example this may occur in sequence specific PCR as discussed below).

In the case where the presence of the polymorphism is being determined in a polynucleotide it may be detected in the double stranded form, but is typically detected in the single stranded form.

The binding agent may be a polynucleotide (single or double stranded) typically with a length of at least 10 nucleotides, for example at least 15, 20, 30, or more polynucleotides. The agent may be a molecule that is structurally similar polynucleotides, comprising units (such as purines or pyrimidines) that are able to participate in Watson-Crick base pairing. The agent may be a protein, typically with a length of at least 10 amino acids, such as at least 20, 30, 50, 100 amino acids. The agent may be an antibody (including a fragment of such an antibody that is capable of binding the polymorphism).

A polynucleotide agent which is used in the method will generally bind to the polymorphism of interest, and the flanking sequence, in a sequence specific manner (e.g. hybridize in accordance with Watson-Crick base pairing) and thus typically has a sequence which is fully or partially complementary to the sequence of the polymorphism and flanking region.

Thus in one method of detection, a binding agent is used as a probe. The probe may be labeled or may be capable of being labeled indirectly. The detection of the label may be used to detect the presence of the probe on (and hence bound to) the polynucleotide or protein of the individual. The binding of the probe to the polynucleotide or protein may be used to immobilize either the probe or the polynucleotide or protein (and thus to separate it from one composition or solution).

In another method of detection, the polynucleotide or protein of the individual is immobilized on a solid support and then contacted with the probe. The presence of the probe immobilized to the solid support (via its binding to the polymorphism) is then detected, either directly by detecting a label on the probe or indirectly by contacting the probe with a moiety that binds the probe. In the case of detecting a polynucleotide polymorphism the solid support is generally made of nitrocellulose or nylon. In the case of a protein polymorphism, the method may be based on an ELISA system, the techniques of which are well known to those of ordinary skill in the art.

Detection methods may be based on an oligonucleotide ligation assay in which two oligonucleotide probes are used. These probes bind to adjacent areas on the polynucleotide which contains the polymorphism, allowing (after binding) the two probes to be ligated together by an appropriate ligase enzyme. However the two probes will only bind (in a manner which allows ligation) to a polynucleotide that contains the polymorphism, and therefore the detection of the ligated product may be used to determine the presence of the polymorphism.

In another detection method the probe is used in a heteroduplex analysis-based system to detect polymorphisms. In such a system when the probe is bound to a polynucleotide sequence containing the polymorphism it forms a heteroduplex at the site where the polymorphism occurs (i.e. it does not form a double strand structure). Such a heteroduplex structure can be detected by the use of an enzyme that is single or double strand specific. Typically the probe is an RNA probe and the enzyme used is RNAse H that cleaves the heteroduplex region, thus allowing the polymorphism to be detected by means of the detection of the cleavage products.

A detection method may be based on fluorescent chemical cleavage mismatch analysis which is described for example in PCR Methods and Applications 3:268-71 (1994) and Proc. Natl. Acad. Sci. 85:4397-4401 (1998).

In one embodiment the polynucleotide agent is able to act as a primer for a PCR reaction only if it binds a polynucleotide containing the polymorphism (i.e. a sequence- or allele-specific PCR system). Hence a PCR product will only be produced if the polymorphism is present in the polynucleotide of the individual. Thus the presence of the polymorphism may be determined by the detection of the PCR product. Preferably the region of the primer which is complementary to the polymorphism is at or near the 3′ end the primer. In one embodiment of this system the polynucleotide agent will bind to the wild-type sequence but will not act as a primer for a PCR reaction.

Detection may be via a Restriction Fragment Length Polymorphism (RFLP) based system. This can be used if the presence of the polymorphism in the polynucleotide creates or destroys a restriction site that is recognized by a restriction enzyme. Thus treatment of a polynucleotide with such a polymorphism will lead to different products being produced compared to the corresponding wild-type sequence. Thus the detection of the presence of particular restriction digest products can be used to determine the presence of the polymorphism.

The presence of the polymorphism may alternatively be determined based on the change that the presence of the polymorphism makes to the mobility of the polynucleotide or protein during gel electrophoresis. In the case of a polynucleotide single-stranded conformation polymorphism (SSCP) analysis may be used. This measures the mobility of the single stranded polynucleotide on a denaturing gel compared to the corresponding wild-type polynucleotide, the detection of a difference in mobility indicating the presence of the polymorphism. Denaturing gradient gel electrophoresis (DGGE) is a similar system where the polynucleotide is electrophoresed through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the Taqman PCR detection system. In brief, this assay uses an allele specific primer comprising the sequence around, and including, the polymorphism. The specific primer is labeled with a fluorescent dye at its 5′ end, a quenching agent at its 3′ end and a 3′ phosphate group preventing the addition of nucleotides to it. Normally the fluorescence of the dye is quenched by the quenching agent present in the same primer. The allele specific primer is used in conjunction with a second primer capable of hybridizing to either allele 5′ of the polymorphism.

In the assay, when the allele comprising the polymorphism is present Taq DNA polymerase adds nucleotides to the nonspecific primer until it reaches the specific primer. It then releases polynucleotides, the fluorescent dye and quenching agent from the specific primer through its endonuclease activity. The fluorescent dye is therefore no longer in proximity to the quenching agent and fluoresces. In the presence of the allele which does not comprise the polymorphism the mismatch between the specific primer and template inhibits the endonuclease activity of Taq and the fluorescent dye in not released from the quenching agent. Therefore by measuring the fluorescence emitted the presence or absence of the polymorphism can be determined.

In another method of detecting the polymorphism, a polynucleotide comprising the polymorphic region is sequenced across the region, which contains the polymorphism to determine the presence of the polymorphism.

Accordingly, any of the following techniques may be utilized in the present methods for genotyping, as is known in the art.

General: DNA sequencing, sequencing by hybridization;

Scanning: PTT (Protein truncation technique), SSCP (single strand conformational analysis), DGGE (denaturing gradient gel electrophoresis), TGGE (temperature gradient gel electrophoresis), Cleavase, Heteroduplex analysis, CMC (chemical mismatch cleavage), enzymatic mismatch cleavage;

Hybridization based: solid phase hybridization (dot blots, MASDA, reverse dot blots, oligonucleotide arrays (chips)); solution phase hybridization (Taqman, Molecular Beacons);

Extension based: ARMS (Amplification Refractory Mutation System), ALEX (Amplification Refractory Mutation System Linear Extension) SBCE (Single Base Chain Extension)

Incorporation based: Mini-sequencing, APEX; (Arrayed Primer Extension)

Restriction enzyme based: RFLP (restriction fragment length polymorphism)

Ligation based: OLA (Oligonucleotide Extension Assay)

Other: Invader (Third Wave Technologies).

In this Example, a method is described for screening a subject diagnosed with IBS or another gastrointestinal disorder treatable by 5HT ligands, to determine the likelihood they will respond in a particular way to treatment with a 5HT ligand, more particularly a 5HT3 antagonist, and more particularly alosetron. Subjects are mammalian, and preferably humans. The method comprises screening the subject for a polymorphism in the 5HTT gene that has previously been associated with a high or low incidence of a particular desirable therapeutic outcome (compared to the incidence in subjects with other genotypes), or associated with a high or low incidence of an undesired side effect (compared to the incidence in subjects with other genotypes), and then classifying the subject as a responder, a partial responder or a non-responder.

Treatment of a subject with a 5HT ligand comprises administration of an effective amount of the pharmaceutical agent to a subject in need thereof. The dose of agent is determined according to methods known and accepted in the pharmaceutical arts, and can be determined by those skilled in the art. A suitable dosage range and plasma concentration for alosetron are provided in the disclosure of U.S. Pat. No. 5,360,800, the entire disclosure of which is hereby incorporated herein by reference.

### Example 2

### Assay of Insertion/Deletion Polymorphism in 5HTT Gene

Genetic samples were obtained from 219 female human subjects enrolled in clinical trials of alosetron for the treatment of IBS. Using PCR technology as is known in the art, an insertion/deletion genetic marker was assayed in the 5-hydroxytryptamine-transporter gene (5HTT gene). The alleles were labeled as “del” (deletion) or “ins” (insertion) resulting in three possible genotypes (del/del; del/ins or ins/ins).

The insertion/deletion marker was in the 5′ untranslated region of the 5HTT gene. The deletion polymorphism (allele “del”) had SEQ ID NO: 1; the insertion polymorphism (allele “ins”) had SEQ ID NO: 2 (insertion shown in bold typeface):

| (SEQ ID NO:1) | |
|---|---|
| ggcgttgccg ctctgaatgc cagccctaac ccctaatgtc cctactgcag cctcccagca | 60 |
| tcccccctgc aacctcccag caactccctg tacccctcct aggatcgctc ctgcatcccc | 120 |
| cattatcccc cccttcactc ctcgcggcat cccccctgca ---------- ---------- | 180 |
| ---------- ---------- ----ccccca gcatcccccc tgcagccctt ccagcatccc | 240 |
| cctgcacctc tcccaggatc tcccctgcaa cccccattat cccccctgca cccctcgcag | 300 |
| tatcccccct gcacccccca gcatcccccc atgcaccccc ggcatccccc ctgcacccct | 360 |
| ccagcattct ccttgcaccc taccagtatt cccccgcatc ccggcctcca agcctcccgc | 420 |
| ccaccttgcg gtccccgccc tggcgtctag gtggcaccag aatcccgcgc ggactccacc | 480 |
| cgctgggagc tgccctcgct tgcccgtggt tgtccagctc agtc cctc 528 | |
| (SEQ ID NO:2) | |
| ggcgttgccg ctctgaatgc cagccctaac ccctaatgtc cctactgcag cctcccagca | 60 |
| tcccccctgc aacctcccag caactccctg tacccctcct aggatcgctc ctgcatcccc | 120 |
| cattatcccc cccttcactc ctcgcggcat cccccctgca ccccccagca tcccccctgc | 180 |
| agccccccca gcatctcccc tgcaccccca gcatcccccc tgcagccctt ccagcatccc | 240 |
| cctgcacctc tcccaggatc tcccctgcaa cccccattat cccccctgca cccctcgcag | 300 |
| tatcccccct gcacccccca gcatcccccc atgcaccccc ggcatccccc ctgcacccct | 360 |
| ccagcattct ccttgcaccc taccagtatt cccccgcatc ccggcctcca agcctcccgc | 420 |
| ccaccttgcg gtccccgccc tggcgtctag gtggcaccag aatcccgcgc ggactccacc | 480 |
| cgctgggagc tgccctcgct tgcccgtggt tgtccagctc agtc cctc 528 | |

The deleted segment comprised nucleotides 161-204 of SEQ ID NO: 2. PCR primer sequences are in underlined typeface.

The present 5HTT genotypes were approximately evenly distributed. Of the 219 subjects genotyped for the 5HTT marker, 71 (32.4%) were del/del 5HTT, 75 (34.2%) were del/ins 5HTT and 73 (33.3%) were ins/ins 5HTT.

The “del” allele represents a deletion of approximately 44 base pairs in the 5′ untranslated region of the 5HTT gene. The del/del genotype results in a lower transcription efficiency, lower production of 5HTT, and reduced basal 5HT re-uptake (compared to the del/ins or ins/ins genotype).

### Example 3

### Correlation of Genotype and Phenotype

The subjects' response to alosetron in the clinical trial setting was reviewed and correlated with genotype. In the double blind, placebo controlled clinical trials; subjects received 12 weeks of treatment with either alosetron or a placebo. A favorable response to alosetron was when a subject reported relief of IBS symptoms during six weeks of the twelve-week trial. The incidence of various other effects, including constipation, was also recorded.

The response of subjects to treatment with alosetron in the clinical trial was stratified according to genotype.

In each of the three 5HTT genotypes alosetron was more effective than placebo in producing relief. However, in the del/del genotype group (homozygous for the deletion polymorphism), an increased incidence of relief of IBS symptoms was seen (increased compared to other 5HTT genotypes). Relief of IBS symptoms with alosetron was achieved in 68% of del/del subjects (21/31); 64% of del/ins subjects (21/33); and 58% of ins/ins subjects (22/38).

The occurrence of constipation during alosetron treatment in the clinical trial was stratified according to genotype. Alosetron treated subjects with the del/del genotype showed a reduced incidence of constipation compared to the del/ins and ins/ins 5HTT-genotype groups. Constipation was reported in 21% of the total group of subjects receiving alosetron (n=102). In del/del subjects (n=31), 4 (13%) reported constipation; in del/ins subjects (n=33), 10 (30%) reported constipation; and in ins/ins subjects (n=38), 8 (21)% reported constipation.

Subjects with the del/del 5HTT genotype showed an increased incidence of favourable therapeutic response, with higher incidence of relief of IBS symptoms and lower incidence of constipation, when compared with subjects with del/ins and ins/ins 5HTT genotypes. The del/del 5HTT genotype can thus be considered as a responder group, leaving the del/ins and ins/ins 5HTT genotype groups being considered as qualified responders or non-responders.

### Example 4

### Genotyping of Individuals for 5HTT Polymorphisms

DNA samples are obtained from a population of subjects with gastrointestinal disease, and genomic DNA is extracted using standard procedures (automated extraction or using kit formats). The genotypes of the subjects, and any control individuals utilized, are determined for polymorphisms within the 5HTT gene sequence, using either PCR, PCR-RFLP, Taqman allelic discrimination assays, or any other suitable technique as is known in the art.

If a specific polymorphism resides in an amplification product that is of sufficient physical size (e.g., an insertion/deletion polymorphism of multiple bases), a simple size discrimination assay can be employed to determine the genotype of an individual. In this case, two primers are employed to specifically amplify the gene of interest in a region surrounding the site of the polymorphism. PCR amplification is carried out, generating products, which differ in length, dependent on the genotype (insertion or deletion) they possess. When subjected to gel electrophoresis, the differently sized products are separated, visualized, and the specific genotypes interpreted directly.

PCR-RFLP (polymerase chain reaction—restriction fragment length polymorphism) assays may also be utilized as is known in the art to detect polymorphisms. For each polymorphic site, a PCR-RFLP assay employs two gene-specific primers to anneal to, and specifically amplify a segment of genomic DNA surrounding the polymorphic site of interest. Following PCR amplification, specific restriction endonuclease enzymes are employed to digest the PCR products produced. The enzyme utilized for an assay is selected due to its specific recognition sequence, which it requires to bind to, and cleave the PCR product in the presence/absence of the polymorphism, yielding fragments diagnostic of the specific base present at the polymorphic site. Following cleavage by the restriction enzyme, gel electrophoresis is employed to separate and visualize the fragments produced.

Taqman assays, as are known in the art, may also be utilized to identify polymorphisms. For each polymorphic site the allelic discrimination assay uses two allele specific probes labeled with a different fluorescent dye at their 5′ ends but with a common quenching agent at their 3′ ends. Both probes have a 3′ phosphate group so that Taq polymerase cannot add nucleotides to them. The allele specific probes comprising the sequence encompassing the polymorphic site and will differ only in the sequence at this site (this is not necessarily true, the allele-specific probes can be shifted relative to each other such that they are not identical in length or composition. However, where they cover the same DNA region they are identical apart from the polymorphic site of interest). The allele specific probes are only capable of hybridizing without mismatches to the appropriate site.

The allele specific probes are used in conjunction with two primers, one of which hybridizes to the template 5′ of the two specific probes, whilst the other hybridizes to the template 3′ of the two probes. If the allele corresponding to one of the specific probes is present, the specific probe will hybridize perfectly to the template. The Taq polymerase, extending the 5′ primer, will then remove the nucleotides from the specific probe, releasing both the fluorescent dye and the quenching agent. This will result in an increase in the fluorescence from the dye no longer in close proximity to the quenching agent.

If the allele specific probe hybridizes to the other allele the mismatch at the polymorphic site will inhibit the 5′ to 3′ endonuclease activity of Taq and hence prevent release of the fluorescent dye.

The ABI7700 sequence detection system is used to measure the increase in the fluorescence from each specific dye at the end of the thermal cycling PCR directly in PCR reaction tubes. The information from the reactions is then analyzed. If an individual is homozygous for a particular allele only fluorescence corresponding to the dye from that specific probe will be released, but if the individual is heterozygous, then both dyes will fluoresce.

The genotypes of the individuals can then be correlated with their phenotypic response to treatment with a 5HT ligand. Responses that vary among the genetic subpopulations are identified as either responders, partial responders or non-responders. Once the non-responder population has been identified, it is assumed that a different genotype is present in that population, which is expressing one or more different proteins that comprise a different biochemical pathway that is the underlying cause of the disease as it is seen in the clinic. Hence the non-responder population becomes the focus of a subsequent clinical trial, in which a drug candidate is administered that has been shown to interact with one or more targets thought to be part of the disease pathway in this population that did not respond to the drug administered in the first trial. If the second trial demonstrates that the second drug candidate elicits a favorable response in the entire population that did not respond to the drug candidate in the first drug trial, then it is apparent that the entire population of patients that started the trials in the first place are now the beneficiaries of safe and effective drug treatments for that clinical definition of disease. It is believed that in many cases, there will be more then two iterations of such clinical trials, reflecting that there are a like number of alternative genotypes that manifest that clinical definition of disease. For example, there may be as many as six distinct genotypes that manifest the disease classified as non-insulin dependant diabetes mellitus. As such, any number of iterations of clinical trials can be run, centered around the method of the invention, that is, that in any given iteration that produces a population of non-responders, the population of non-responders represents a whole new group of patients that likely have a different genotype that is treatable by a drug that is different from the drug tested in the previous iteration of clinical trial.

1. A method of using biological markers for the discovery and development and prescribing of medicines, such method comprising the steps of:
(a) obtaining a biological sample from a patient;
(b) delivering the sample of step (a) to a centralized analysis and storage facility;
(c) genotyping the sample of step (a) at the facility of step (b);
(d) electronically providing the genotype analysis of step (c) by said facility of step (b) back to said patient upon request by said patient or said patient's healthcare provider in order to enable said healthcare provider to form a judgement as to the most appropriate drug to administer to said patient in view of said patient's genotype;
(e) contemporaneously electronically providing the genotype analysis of step (c) by said facility of step (b) to a peer review body for data analysis and then transmitting such analyzed data to a database so as to enable discovery of one or more associations between a given genotype and a given response to a given drug;
(f) contemporaneously electronically providing the reviewed data and/or the discovered associations of step (e) back to the facility of step (b); and
(g) contemporaneously electronically providing the reviewed data and/or discovered associations of step (e) by the facility of step (b) to one or more healthcare providers upon request by a healthcare provider in order to enable said healthcare provider to form a judgement as to the most appropriate drug to administer to a given patient having a genotype that is present in one or more of said discovered associations.

2. A method of using biological markers for optimizing the delivery of individual patient therapeutic intervention in the management of disease, such method comprising the steps of:
(a) obtaining a biological sample from a patient;
(b) delivering the sample of step (a) to a centralized analysis and storage facility;
(c) genotyping the sample of step (a) at the facility of step (b);
(d) electronically providing the genotype analysis of step (c) by said facility of step (b) back to said patient upon request by said patient or said patient's healthcare provider in order to enable said healthcare provider to form a judgement as to the most appropriate drug to administer to said patient in view of said patient's genotype;
(e) contemporaneously electronically providing the genotype analysis of step (c) by said facility of step (b) to a peer review body for data analysis and then transmitting such analyzed data to a database so as to enable discovery of one or more associations between a given genotype and a given response to a given drug;
(f) contemporaneously electronically providing the reviewed data and/or the discovered associations of step (e) back to the facility of step (b); and
(g) contemporaneously electronically providing the reviewed data and/or discovered associations of step (e) by the facility of step (b) to one or more healthcare providers upon request by a healthcare provider in order to enable said healthcare provider to form a judgement as to the most appropriate drug to administer to a given patient having a genotype that is present in one or more of said discovered associations.

3. The method as claimed in claim 2, wherein said biological sample collection; sample storage, data analysis and data reporting are conducted globally and not limited by territorial boundaries.

4. The method as claimed in claim 2, wherein samples are delivered to one or more centralized facilities.

5. The method as claimed in claim 4, wherein said facility or facilities operates said analysis and reporting functions.

6. The method as claimed in claim 4, wherein said facility or facilities is managed by a consortium.

7. The method as claimed in claim 6, wherein said consortium is comprised of members selected from the group consisting of pharmaceutical companies, biotechnology companies, academic institutions, healthcare providers, insurers, patients, patient advocacy groups, regulators and governmental bodies.

8. The method as claimed in claim 2, wherein said process of sample collection is under the control of said patient.

9. The method as claimed in claim 2, wherein a biological sample is taken from a patient one time only.

10. The method as claimed in claim 2, wherein said biological sample is analyzed against a panel of markers defined by a specific medicine response profile protocol.

11. The method as claimed in claim 2, wherein said healthcare provider is enabled to form a judgement as to the most appropriate method for therapeutic intervention for a patient who is deemed to be at risk of an adverse event based on their medicine response profile test result.
